(19) United States
(12) Patent Application Publication
(10) Pub. No.: US 2025/0090855 A1
(43) Pub. Date: Mar. 20, 2025

(54) DEVICE AND METHOD FOR TRANSCRANIAL MAGNETIC STIMULATION

(71) Applicant: BRAINSWAY LTD, Jerusalem (IL)

(72) Inventors: Yiftach ROTH, Rechelim (IL); Gaby S. PELL, Jerusalem (IL); Abraham ZANGEN, Jerusalem (IL)

(21) Appl. No.: 18/728,080

(22) PCT Filed: Jan. 11, 2023
(86) PCT No.: PCT/IL2023/050034

§ 371 Date: Jul. 11, 2024

Related U.S. Application Data
(60) Provisional application No. 63/298,428, filed on Jan. 11, 2022.

(51) Int. Cl. A61N 2/02; A61N 2/00

(52) U.S. Cl. CPC A61N 2/02; A61N 2/006

(57) ABSTRACT

A TMS coil assembly for magnetic stimulation of a brain region of a treated subject. The TMS coil assembly comprising one or more coil assemblies, each of which comprising adjacently located, or spaced-apart, flexible winged-coil structures configured to adjustably deform along a tilt axis passing between the winged-coil structures to apply a desired tilt angle between the winged-coil structures for them to conform to curved surfaces of a head of a treated subject and bring substantial loop portions of the winged-coil structures into direct contact with the curved surfaces of the head, to thereby maximize the loop portions that are tangential to the curved surfaces of the head and minimize the loops portion that are non-tangential to the curved surfaces. A fastening arrangement can be used to maintain the desired tilt angle between the winged-coil structures when placed on the head of the treated subject during a TMS procedure, to thereby maximize strength of electric field thereby induced into the head.

### TECHNOLOGICAL FIELD

The present disclosure generally relates to brain stimulation devices and methods, and in particular to transcranial magnetic stimulation (TMS) device for stimulating selected brain regions.

### BACKGROUND

This section intends to provide background information concerning the present application, which is not necessarily prior art.

Transcranial magnetic stimulation (TMS) is a noninvasive technique used to apply brief electromagnetic pulses to the brain, to thereby activate neuronal structures thereof. The pulses are administered by a stimulator configured to pass high electric currents through an electromagnetic coil externally placed on/near a head region of the patient (for example, placed on the scalp for brain treatment), thereby inducing electrical currents in the underlying tissue and producing a localized axonal depolarization. This technique has become a major tool in central nervous system research, as well as an established treatment option for various psychiatric disorders and a potentially promising treatment option for various neurobehavioral and neurological disorders.

Most known TMS coils stimulate superficial brain regions in the brain cortex, but the rate of decay of the magnetic and electric fields induced by such TMS coils as a function of distance from the coil is high. Hence the efficacy of affecting deeper neuronal structures is typically low with the conventional TMS coils. Stimulating deeper neuronal structures may be feasible if the intensity of the induced field is greatly increased. Yet operation at such increased intensity may increase the risk for seizures and for physiological damage to the brain tissue.

A method for deep brain TMS with minimal stimulation of superficial regions is disclosed in U.S. Pat. No. 7,407,478, the disclosure of which is incorporated herein by reference, wherein deep brain stimulation is made possible while minimizing side effects. The device described therein includes a base and an extension portion, the base having individual windings for individual paths of current flow, and the extension portion designed so as to minimize unwanted stimulation of other regions of the brain.

The most widely used TMS coil is a flat figure-8 coil (also referred to herein as butterfly-shaped coil) e.g., having two spiral-shaped coil wings arranged in a common plane. The flat butterfly-shaped coil is placed over the cortex and only the is central segment contacts the head/scalp of treated subject. At that point, underneath the central segment of the butterfly-shaped coil, the maximal electric field is induced, and neural structures in this region are responsively stimulated. Yet, studies with volume conductors having flat (Branston N. M. et al, “Magnetic stimulation of a volume conductor produces a negligible component of induced current perpendicular to the surface”, J. Physiol. (Lond) 1990; 423:67; Roth B. J. et al, “A model of the stimulation of a nerve fiber by electromagnetic radiation”, IEEE Trans. Biomed Eng. 1990; 37:588-597; Tofts P. S. “The distribution of induced currents in magnetic stimulation of the nervous system”, Phys. Med. Biol. 1990; 35:1119-28; Tofts P. S. et al, “The measurement of electric field, and the influence of surface charge, in magnetic stimulation”, Electroencephalogr. Clin. Neurophysiol. 1991; 81:238-9) and spherical (Branston N. M. et al, “Analysis of the distribution of currents induced by a changing magnetic field in a volume conductor”, Phys. Med. Biol. 1991; 36:161-8; Cohen D. et al, “Developing a more focal magnetic stimulator. Part I: some basic principles”, J. Clin. Neurophysiol. 1991; 8:102-11; Eaton H. “Electric field induced in a spherical volume conductor from arbitrary coils: application to magnetic stimulation and MEG”, Med. Biol. Eng. Comput. 1992; 30:433-40) geometries have demonstrated that coil elements perpendicular to the brain surface induce accumulation of surface charge, which leads to complete cancellation of the perpendicular component of the induced field at all points within the tissue. In addition, the electrical field in any other direction is considerably reduced.

Thus, there is a need for TMS coil designs having a minimal number of coil elements which are perpendicular to the surface of the skull of the treated subject, in order to reduce the electrostatic charge accumulation and enhance the efficacy of magnetic coupling of the coil with the brain tissue. The coils should induce the desired electric field intensity in the relevant brain tissue, which will be feasible for neuronal stimulation with available TMS stimulators for most of the population. The stimulation intensity is routinely calibrated individually for each subject based on the subject's motor threshold. Hence the coil efficiency should guarantee that the motor threshold and stimulation intensity for most of the relevant population is within an acceptable range with respect to available stimulators' power outputs.

The coils' design thus need to be efficient with respect to energy consumption, coil heating rate, compact size and ease of operation.

A method for application of rotating fields by TMS coils is disclosed in U.S. Pat. No. 9,067,052, wherein two coils are placed perpendicular to each other, and wherein an alternating electric current pulse is passed through each coil. The alternating electric current pulses of the two coils are not simultaneous but have a time delay between the operation of one coil and the operation of the second coil, so that there is a phase between the currents of the two coils, and hence also between the pulses of the electric fields induced in the brain tissue by the coils. In case where the phase is 90 degrees, a rotating field is induced inside the brain. In general, neurons with axons parallel to the induced electric field may be stimulated. Hence, with this setting, neurons in the brain region aligned in various orientations may be stimulated.

### GENERAL DESCRIPTION

There is a need in the art for a TMS device configured for efficient stimulation of deep neural structures in the brain of a treated subject, and which can also be adjustable so as to fit to the size and shape of the skull/head of the treated subject. The present application provides electromagnetic coils designed to induce magnetic fields into brain tissue of a treated subject.

In order to optimize the efficacy of stimulation of nerve tissue in the desired brain regions/portions, it is desirable to minimize magnitude of non-tangential components of the induced electric field. In order to effectively and efficiently stimulate deep neuronal tissue, the coil configurations disclosed herein are designed to provide high electric field magnitude in a direction tangential to the head/skull, while minimizing electric fields in all non-tangential directions.

Generally, the ability to create action potential responses in neurons depends mainly on two properties, size, and orientation of the neurons. The dependence on orientation arises since the magnetic stimulation of a neuron occurs at the axon, whose projection along the induced electric field is the relevant parameter for achieving excitation. Typically, orientation of neurons in deep brain tissues is random, namely, neurons are aligned in different orientations. Therefore, by inducing a rotating electric field whose orientation affects a wide range of angles during a single pulse, many axons are excited all at once and thus many neurons are stimulated. The rotating field can be induced utilizing any of the techniques disclosed in U.S. Pat. No. 9,067,052, the disclosure of which is incorporated herein by reference. There is provided, in accordance with possible embodiments hereof TMS coil configurations for transcranial magnetic stimulation. The TMS coil disclosed herein generally has one or more butterfly-shaped coils, each having two or more coupled winged-coil structures designed for enhanced flexibility.

The term winged-coil structure used herein to generally refer to a coil structure comprising an arrangement of one or more EM coil-loops that are located adjacent (in close-proximity) to, or spaced apart from, another arrangement of one or more EM coil-loops i.e., another winged-coil structure. It is noted that the butterfly-shaped coils disclosed herein are not required to be symmetrical i.e., the EM coil-loops can be arranged differently in the adjacently located or spaced-apart winged-coil structures e.g., the EM coil-loops in each winged-coil structure can have different geometrical shapes, different number of loops, and/or wires having different cross-sectional areas and/or geometrical shapes. In addition, in possible embodiments the EM coil-loops of the of the winged-coil structures are not necessarily electrically coupled or connected to each other.

Accordingly, in some embodiments the butterfly-shaped coil has a figure-8 shape, having two adjacently located winged-coil structures. In other possible embodiments the winged-coil structures of the butterfly-shaped coil have a defined distance (e.g., between 10 to 70 mm) separating between them. The two winged-coil structures of the butterfly-shape coil are made flexible in some embodiments, and can be hinged one to the other such that they can be moved/deformed one with respect to the other to conform the shape of the head of the treated subject. This way, the geometry of the TMS coil can be adjusted to the curvature of the head of the treated subject e.g., to establish a grip thereover, so that many, all, or most, of the loop elements of the winged-coil structures touch the head of the treated subject i.e., the coil elements touch the hair and/or scalp of the treated subject.

In the embodiments disclosed herein segments/portions of the individual loop(s) of the winged-coil structures that are located in the center of the butterfly-shaped coil(s) are carrying electric currents in the main direction of the respective winged-coil structures and thus referred to herein as main loops' segments or portions. The embodiments disclosed herein are configured such that orientations of the main loops' segments are all, or mostly, tangential to the head curved surface therebeneath (such as a portion of the skull), at all or a substantial part of their path. In order to optimize the efficacy of neural activation in the target brain structures located under the central segment of the butterfly-shaped coil(s), it is desirable to minimize the non-tangential components of the induced electric field. Since the orientation of the electric field induced by any coil element is parallel to the orientation of the coil's elements carrying the alternating electric currents, it is desirable to minimize the portions of coil's elements that are non-tangential to the head (skull) of the treated subject.

Segments/portions of the butterfly-shaped coils that carry electric current in a direction opposite to the main direction, are positioned at the lateral sides/externus of the two winged-coil structures, and referred to herein as return loops' segments or portions. In possible embodiments the return loops' segments are also made flexible so as to conform to the curved surface of the head, or head portion, of the treated subject. Optionally, but in some embodiments preferably, only the return loops' segments of winged-coil structure are configured to deform and adapt to a geometrical shape of a portion of the head of the treated subject, while the geometrical shape of the main loops' segments remain substantially unchanged.

In some embodiments the winged-coil structures of the butterfly-shaped coil(s) can be locked in a flat position/conformation such that all of the loops' elements of the winged-coil structures are substantially located in a common plane. In this flat configuration the coil functions similar to a conventional/standard figure-8 shape TMS coil.

In some embodiments the winged-coil structures of the butterfly-shaped coil(s) can be changed between a locked and released states, so that in the released state they can be moved one with respect to the other and conform to the curvature of the head, or a portion of the head, of the treated subject. One or more releasable connectors and/or elastic/adjustable straps can be used to hold the winged-coil structures in a desired angle therebetween in locked state of the butterfly-shaped coil(s). The TMS coil may include one or more head straps (also referred to herein as securing means), and/or other attachment means, configured to secure the TMS coil to the head of the treated subject, and/or enable firm attachment of all, or most, of the coil loops' elements at the desired location and/or tilt/angle, during a TMS treatment session conducted therewith.

In some embodiments the TMS coil device comprises an array of crossing coils structure, wherein each crossing coil structure has a deployment axis crossing a deployment axis of at least another coil structure of the TMS coil device. For example, in some embodiments the crossing coils structure forms an equiangular TMS coil structure, wherein the angle between the deployment axes of each pair of adjacently located crossing coils is substantially 180°/n, wherein n≥2 is the (integer) number of crossing coils in the TMS coil structure.

Optionally, but in some embodiments preferably, the array of crossing coils comprises a lower flexible butterfly-shaped coil, and an upper butterfly-shaped coil having a desired angle of orientation of its winged-coil structures relative to an orientation of the winged-coil structure of the lower butterfly-shaped coil i.e., the angle between the deployment axes of the butterfly-shaped coils equals, or smaller than, 90°. In some embodiments the lower flexible butterfly-shaped coil is carrying an upper flexible butterfly-shaped coil, and the angle between the deployment axes of the winged-coil structures of the lower and upper butterfly-shaped coils is about 90 degrees.

Each one of the crossing coils (e.g., the lower and upper flexible butterfly-shaped coils) can be electrically connected to a respective channel of a multi-channel TMS stimulator, or alternatively, to different stimulator units. The timing of the operation of the crossing coils can be controlled to implement a desired TMS stimulation protocol. For example, if the coils of a two crossing coils TMS coil structure are operated with a time delay effecting a 90 degrees phase shift, a rotating field is induced in the underlying brain tissue and neurons in various orientations may be stimulated.

In some possible embodiments crossing coils (e.g., the flexible butterfly-shaped coils) are made from an electrically conducting wire (e.g., made of copper). In some embodiments the electrically conducting wires have cross-sectional area of about 7 to 10 mm², wound to form a coil comprising a plurality of loop windings electrically connected in series. The electrically conducting wires are preferably electrically insulated. In some embodiments the windings are made of several wires twisted or packed together.

In some embodiments there are several layers of electrical insulation coating the electrically conducting wires of the coils. For example, the electrically conducting wires may be electrically insulated with at least one layer of insulation, such as for example sintered PTFE. In addition, all the electrically conducting wires may be surrounded by one or more layers of shield which give electric insulation. Such shield may for example include Tape-Wrapped-Sintered CR PTFE and/or Double SPC Braid Shield.

In some embodiments the loop windings configuration of the coil (e.g., of the winged-coil(s)) is held by a support structure, which on one hand maintains a generally fixed shape of the elongated coil configuration, and on the other hand permit a certain degree of freedom of movement and flexibility.

In some embodiments there is one degree of freedom in adjusting the angle between the coil segments/portions extending along, or with respect, to the deployment axis of the coil (e.g., the wings of at least one of the flexible winged-coil structures). In other possible embodiments additional degrees of freedom are provided, such as flexibility of the coil segments/portions (e.g., the wings of the butterfly-shaped coil elements), which enables them to conform to a curved shape of a human head, or to at least some portion of the head.

The number, length, configuration and packing parameters of the windings of the flexible coil(s) are configured to obtain coil inductance of a desired range. Usually, the desired inductance range of each of the crossing coils (e.g., a flexible butterfly-shaped coil) is between 15 to 30 microHenri. Too high inductance may reduce coil efficacy, increase pulse width and is often associated with increased coil resistance, energy consumption and coil heating. Too small inductance may lead to fast rate of change of the electric current which may damage components of the stimulator.

In some embodiments neuronavigation tools are integrated into the TMS device. Neuronavigation is important to enable accurate placement of the coils at the desired location over the head, and in order to perform repeatable and accurate stimulation of the desired brain target. There are few types of neuronavigation techniques that can be used. One neuronavigation technique is based on an optical neuronavigation approach in which sensors are placed on at least one of the TMS coils and on the head of the treated subject.

For example, in possible embodiments a camera with transmitter and receiver, usually in the infrared (IR) range, is arranged to constantly track the locations and orientations of the sensors. The locations of the sensors can be registered in association to a brain mapping e.g., obtained based on either standard MRI or personal MRI of the individual subject being treated. A processor can be used to integrate all the information, the sensors' locations along with MRI-based image of the brain, which are presented on a display/screen, and the system can give instructions for manually navigating the TMS coil(s) for placement on the target position of the head of the treated subject based on the registered sensors' locations and the brain mapping. In addition, the system can constantly track the location of the TMS coils on the head, give warnings in case of deviation from the allowed range and ensure accurate coils' positioning throughout the treatment session.

Another neuronavigation approach is based on electromagnetic (EM) tracking. In this approach, EM sensors are placed on the TMS coil(s) and on the head of the treated subject, and serve as localization points for the instrument in 3D space. A field generator or transmitter is used to emit a low-intensity, varying EM field that establishes the measurement volume. Small currents are induced inside the EM sensors when they enter the EM field. These induced currents are relayed to the sensor interface unit (SIU), wherein they are amplified and digitized to generate measurement data/signals. The measurement data/signals are transmitted to the system's control unit (SCU), which calculates the position and orientation of each EM sensor on the head of the treated subject e.g., as a transformation.

In another version, the functions of the SIU and SCU are performed by an electronic unit, which calculates sensor tracking data as positions and rotational matrix. The tracking data is communicated to an application interface for real-time manual navigation of instruments relative to patient image sets, based on either standard MRI or personal MRI of the individual subject being treated. An advantage of the EM-based tracking method is that no line of sight is needed, as opposed to the optical method which requires line of sight between the camera and the sensors.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. In case of conflict, the specification, including definitions, will control. In addition, the materials, methods, and examples are illustrative only and not intended to be limiting.

In one aspect there is provided a TMS coil assembly for magnetic stimulation of a brain region of a treated subject. The TMS coil assembly comprises one or more coil assemblies, each one of the coil assemblies comprising adjacently located, or spaced-apart, flexible winged-coil structures configured to adjustably deform along a tilt axis passing between the winged-coil structures to apply a desired tilt angle between the winged-coil structures for them conform to curved surfaces of a head of a treated subject and bring substantial loop portions of the winged-coil structures into direct contact with the curved surfaces of the head, to thereby maximize the loop portions that are tangential to the curved surfaces of the head and minimize the loops portion that are non-tangential to the curved surfaces. A fastening arrangement having a coil retaining structure attached to each butterfly-shaped coil can be used to maintain the desired tilt angle between the winged-coil structures butterfly-shaped coils when placed on the head of the treated subject during a TMS procedure, to thereby maximize strength of electric field thereby induced into said head. Optionally, the desired tilt angle is a flat angle.

The TMS coil assembly is configured in some embodiments to define a field application axis passing between the winged-coil structures. The tilt axis and the field application axis can be substantially parallel or coincide. Optionally, the TMS coil assembly configured to define a central interaction zone, with respect to the field application axis, in which additive electric and magnetic fields generated by the winged-coil structures are of maximal strength. In possible applications the TMS coil assembly comprises a locking mechanism configure to lock the winged-coil structures at in the desired tile angle applied by the fastening arrangement.

In some embodiments the fastening arrangement comprises one or more coil retaining structures attached to the winged-coil structures and configured to facilitate adjustment of the tilt angle. Optionally, but is some embodiments preferably, the one or more coil retaining structures are hinged with one or more pivots. One or more levering members, each coupled to a coil holder apparatus and attached to at least one of the coil retaining structures, can be used to facilitate setting of the desired tilt angle between the winged-coil structures. A locking mechanism can be used with this configuration to maintain the levering members locked with respect to the coil holder apparatus so as to prevent further tilting of the winged-coil structures.

The TMS coil assembly may comprise one or more releasable connectors, each configured to couple one of the levering members to the coil holder apparatus. Each releasable connector can be connected to a respective levering member by a respective elastic and/or adjustable strap.

Securing means are provided in some embodiments to attach the TMS coil assembly to the head of the treated subject. The securing means may comprise head straps configured to secure the TMS coil assembly to the head and bring substantial portion of their loops into direct contact therewith.

The TMS coil assembly can be configured with several degrees of flexibility of the winged-coil structures/lobes for allowing the loops of the coil to conform to the head of the treated subject and bring substantial portions of the loops into direct contact therewith. Optionally, the loops of the winged-coil structures are configured to flexibly conform and contact the head in all of the coil portion facing the head.

In possible embodiments the winged-coil structures have a generally circular shape, or a generally elliptical shape, or a generally rectangular shape.

In another aspect there is provided a TMS coil array for magnetic stimulation of a brain region of a treated subject. The TMS coil array comprising two or more of the TMS coil assemblies according to any of the embodiments disclosed herein, each one of the TMS coil assemblies having a deployment axis being substantially perpendicular to its tilt axis, where the deployment axis of each one of the two or more TMS coil assemblies crosses the deployment axis of at least another one of the TMS coil assemblies. In some embodiments the TMS coil array comprises two of the TMS coil assemblies connected to two different sides of a single fastening arrangement. Optionally, the angle between the tilt axes of the TMS coil assemblies is about 90 degrees.

In some embodiments each one of the two or more TMS coil assemblies has a central interaction zone and wherein the TMS coil array is configured such that the interaction zones of the two or more TMS coil assemblies substantially coincide.

Optionally, each one of the TMS coil assemblies is electrically connected to a respective independent channel of a stimulator device configured for controlling the timing of operation of the TMS coil assemblies. In possible embodiments the TMS coil array is configured for operation of a phase difference between electric currents of at least two of the TMS coil assemblies, to thereby induce a rotating field in the brain tissue.

In possible embodiments each or some wires of the TMS coil assemblies are electrically insulated separately. Optionally, one or more layers of an electric insulation shield can be used to surround all wires of the TMS coil assemblies.

The TMS coil array is configured in some embodiments for neuronavigation of position of the coil assemblies on the head. The neuronavigation can be based on optical and/or electromagnetic tracking.

In yet another aspect there is provided a method for treating a neurophysiological condition. The method comprising placing at least one TMS coil assembly having winged-coil structures on a head portion of a treated subject, setting a tilt angle between the winged-coil structures for them to conform to curved surfaces of the head so as to bring substantial portions of their loops into direct contact with the head, and passing electric currents in said at least one TMS coil assembly to stimulate a region of the brain of the treated subject. The method may comprise changing the TMS coil assembly into a released state before the setting of the tilt angle, and thereafter changing the TMS coil assembly into a locked state for preventing further tilting between the winged-coil structures.

In still yet another aspect there is provided a method for treating a neurophysiological condition. The method comprising placing the TMS coil assembly, or the TMS coil array, of any one of the embodiment disclosed herein, on a head of a treated subject and setting a tilt angle between at least two winged-coil structures thereof so as to conform to a curved surface areas of said head, and passing electric currents in the TMS coil assemblies of said TMS coil array to stimulate a region of a brain of the subject. The neurophysiological condition can beat least one of the following: depression, bipolar disorder, schizophrenia, PTSD, Parkinson's disease, dystonia, movement disorder, Altzheimer's disease, mild cognitive impairment, autism, Asperger's syndrome, multiple sclerosis, ALS, Tourette's syndrome, blepharospasm, stroke, chronic pain, eating disorder, obesity, anorexia nervosa, bulimia, any addiction including smoking addiction, drug addiction, alcoholism or gambling, ADHD, OCD, epilepsy, migraine, tinnitus.

### BRIEF DESCRIPTION OF THE DRAWINGS

In order to better understand the subject matter disclosed herein and to exemplify how it may be carried out in practice, embodiments will now be described, by way of non-limiting example only, with reference to the accompanying drawings. Features shown in the drawings are meant to be illustrative of only some embodiments of the invention, unless otherwise implicitly indicated. In the drawings like reference numerals are used to indicate corresponding parts, and in which:

FIGS. 1A to 1G schematically illustrate a TMS coil device comprising according to some possible embodiments a flexible circular butterfly-shaped coil, wherein FIG. 1A is a perspective illustration of a circular butterfly-shaped coil with an angle between the two wings of the coil, FIG. 1B is a top view of the circular butterfly-shaped coil configured to form a flat/planar wings constellation that is similar to a standard figure-8 shape coil, FIG. 1C is an illustration of the flexible circular butterfly-shaped coil attached to the head of a treated subject at a medial position with the winged-coil structure conforming to the shape of the head/skull, and FIG. 1D is an illustration of a flexible circular butterfly-shaped coil attached to the head of the treated subject at a lateral position wherein the center of the butterfly-shaped coil is approximately above the right hand motor cortex representation at the left hemisphere, with the coil elements/wings conforming to the shape of the head, FIG. 1E illustrates a circular butterfly-shaped coil with means and mechanisms for enabling to lock it in a flat/planar wings constellation, FIG. 1F shows a coil in a case, with the means enabling to lock it in a flat/planar wings constellation, and FIG. 1G illustrates a flexible circular butterfly-shape coil over a human head with means and mechanisms enabling to conform its wings to curvature of the head, and to attach it to the head with a suitable angle between the wings;

FIGS. 2A to 2D schematically illustrate a TMS coil device comprising according to some possible embodiments an elliptical butterfly-shaped coil, wherein FIG. 2A is a top view of the elliptical butterfly-shaped coil configured in some embodiments to form a flat wings constellation that is similar to a standard figure-8 shape coil, FIG. 2B is a side view of an elliptical butterfly-shaped coil with an angle between the two wings FIG. 2C illustrates the flexible elliptical butterfly-shaped coil attached to the head of the treated subject at a medial position with the coil/wing elements conforming to the shape of the head, and FIG. 2D illustrates the flexible elliptical butterfly-shaped coil attached to the head of the treated subject at a lateral position, wherein the center of the coil is approximately above the right hand motor cortex representation at the left hemisphere, with the coil elements conforming to the shape of the head;

FIGS. 3A to 3E schematically illustrate a TMS device comprising according to some possible embodiments an array of flexible crossing coils, wherein FIGS. 3A and 3B are top and bottom perspective illustrations of the array of flexible crossing coils comprising a lower butterfly-shaped coil and an upper butterfly-shaped coil in perpendicular orientation one to the other, FIG. 3C illustrates the array of flexible crossing coils with electromagnetic neuronavigation sensors coupled to the two wings of the lower flexible crossing coil, and FIG. 3D illustrates an array of crossing coils having a lower flexible elliptical butterfly-shaped coil and an upper flexible circular butterfly-shaped coil, arranged in a perpendicular orientation one with respect to the other and attached to the head of the treated subject at a medial position with the coils elements conforming to the shape of the head, and FIG. 3E illustrates the array of crossing coils attached to the head of the treated subject at a lateral position where the center of the crossing coils structure is approximately above the right hand motor cortex representation at the left hemisphere, with the coils elements/wings conforming to the shape of the head; and

FIGS. 4A to 4C schematically illustrate coil stimulation schemes according to some possible embodiments.

### DETAILED DESCRIPTION OF EMBODIMENTS

One or more specific embodiments of the present disclosure will be described below with reference to the drawings, which are to be considered in all aspects as illustrative only and not restrictive in any manner. In an effort to provide a concise description of these embodiments, not all features of an actual implementation are described in the specification. Elements illustrated in the drawings are not necessarily to scale, or in correct proportional relationships, which are not critical. Emphasis instead being placed upon clearly illustrating the principles of the invention such that persons skilled in the art will be able to make and use the disclosed coil assemblies, once they understand the principles disclosed herein. This invention may be provided in other specific forms and embodiments without departing from the essential characteristics described herein.

In a broad aspect the present disclosure aims to combine techniques for inducing a rotating electromagnetic field inside the head of the treated subject, with flexible coil designs configured to minimize, or altogether eliminate, coil elements/portions which are perpendicular to the surface of the head of the treated subject.

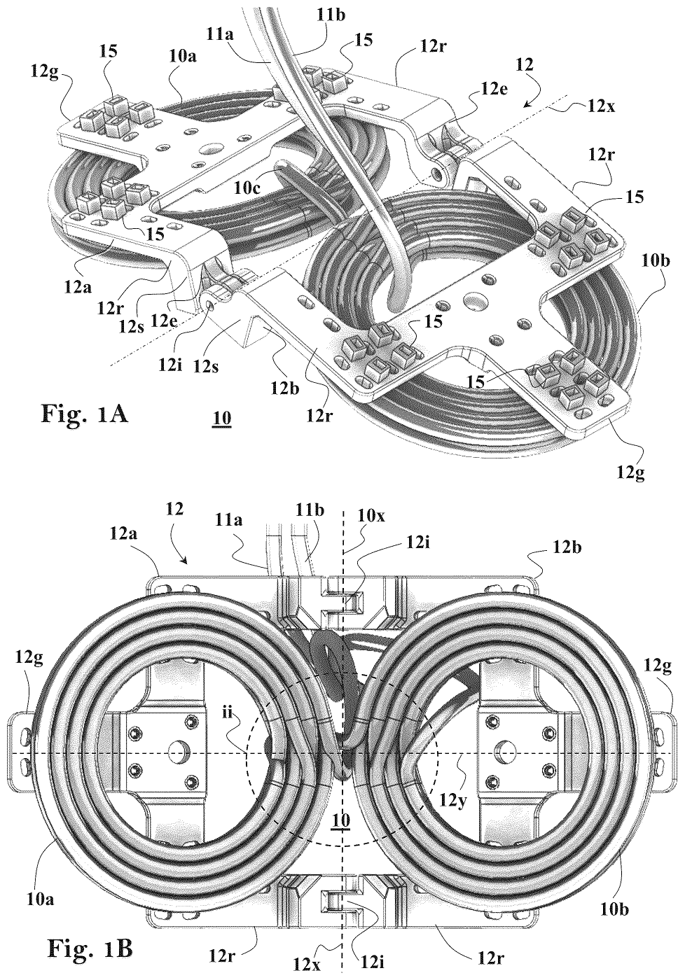

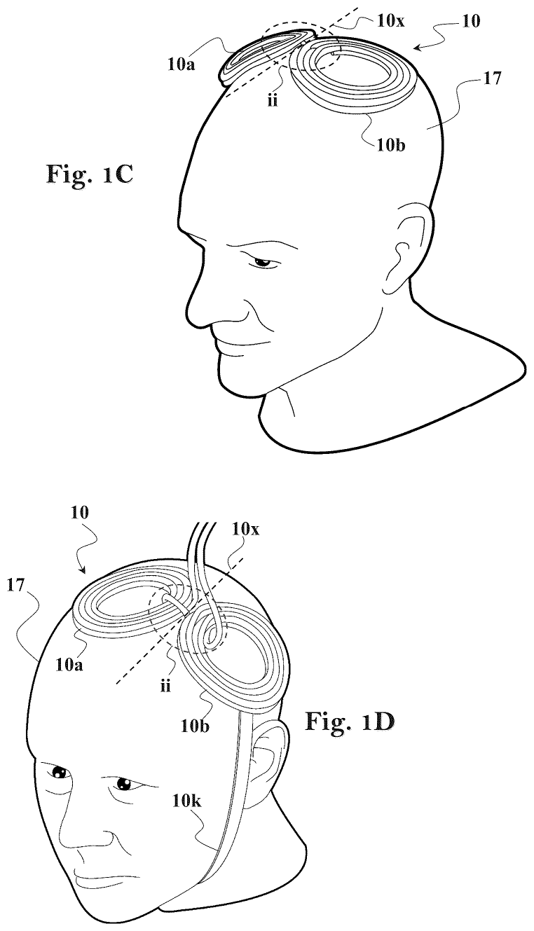

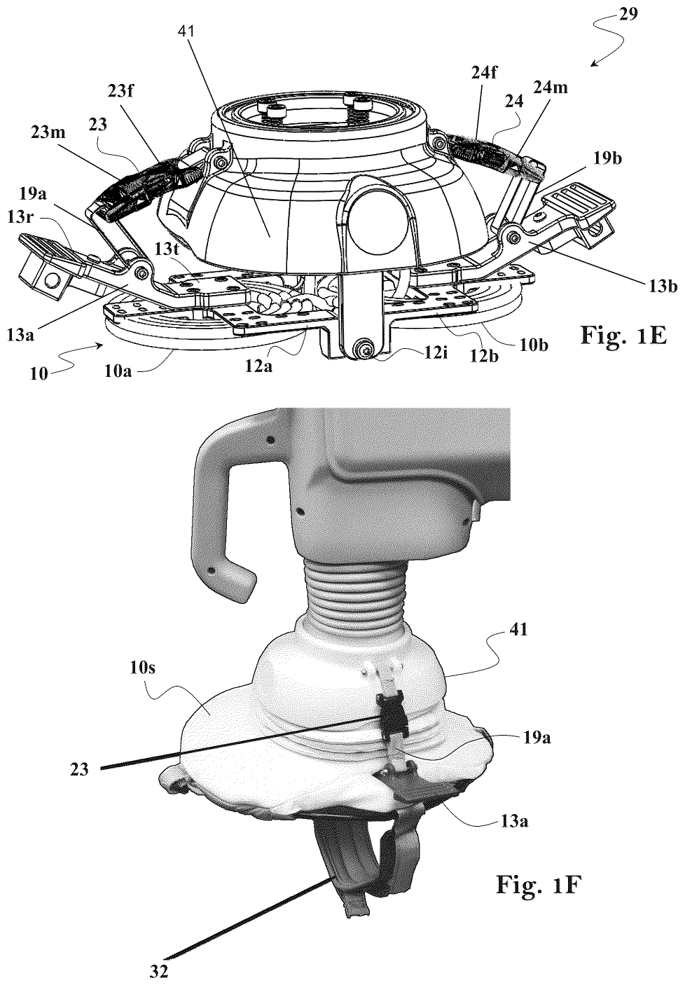

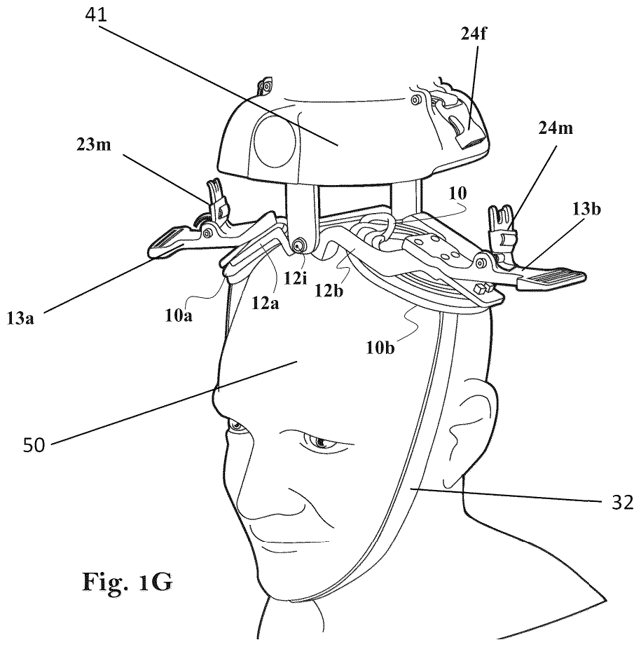

Reference is made to FIGS. 1A and 1B, schematically illustrating, respectively top-perspective and bottom views of a butterfly-shaped electromagnetic coil assembly 10 according to some possible embodiments. The electromagnetic butterfly-shaped coil assembly 10 includes two spaced apart winged-coil structures/lobes 10a and 10b comprising a plurality of loops, each winged-coil structure/lobe 10a, 10b forming a substantially planar circular-shaped coil structure coupled to a support unit comprising an adjustable/flexible fastening arrangement 12 mechanically connecting between the two spaced apart winged-coil structures/lobes of loops 10a and 10b. The fastening arrangement 12 is configured in some embodiments for varying an angle between planes of the two spaced apart winged-coil structures/lobes 10a and 10b, so as to fit/conform the coil assembly 10 to the size/shape of the head/skull of the treated subject.

In some possible embodiments, the fastening arrangement 12 comprises a pair of “Y”-shaped coil retaining structures, 12a and 12b. Each of the “Y”-shaped coil retaining structures 12a and 12b has a respective leg portion 12g and two arm portions 12r extending therefrom. The arm portions 12r can extend diagonally in sideway directions from leg portion 12g of each “Y”-shaped coil retaining structure 12a, 12b to form a three-vertex star structure. In this specific and non-limiting example, the arm portions 12r extend in opposing sideway directions from their respective leg portion 12g, to extend along a diameter of their respective winged-coil structure/lobe 10a, 10b, and therefrom they extend axially to engage with the arm portions 12r of the other winged-coil structure/lobe of the coil structure 10.

The winged-coil structures/lobes 10a and 10b of the coil structure 10 are secured to the leg 12g and arms 12r, for example, via fasteners 15 e.g., cable-tie/zip-tie. Each of the arm portions 12r includes a respective shoulder portion 12s pivotally connected to an arm portion 12r of the wing/lobe e.g., via hinge 12i configured to enable angular movement of the “Y”-shaped coil retaining structures 12a and 12b one with respect to other. Accordingly, an angle between the “Y”-shaped coil retaining structures 12a and 12b (and consequently between the planes of the two spaced apart winged-coil structures/lobes 10a and 10b) can be manipulated, i.e., increased or decreased, so as to vary angular orientation of the two spaced apart winged-coil structures/lobes 10a and 10b (planes thereof) with respect to each other. This way the coil assembly 10 can be adjusted to snugly fit/conform to the size of the head/skull of the treated subject such that a significant amount the bottom-side windings each of its wings/lobes 10a, 10b contact the head of the treated subject.

FIG. 1A shows the coil assembly 10 in a slanted conformation in which an obtuse angle is formed between the winged-coil structures/lobes 10a, 10b of the coil assembly 10, as they are angularly tilted about their tilt axis 12x defined by the hinges 12i. As seen, each shoulder portion 12s can extend vertically downwardly or upwardly from the free end of its respective arm portion 12r to define an engagement face 12e for the respective hinge 12i pivotally connecting with an arm portion 12r of the other “Y”-shaped coil retaining structure.

Optionally, and in some embodiments preferably, each of the substantially planar winged-coil structures/lobes 10a, 10b of the coil structure 10 is made from a single continuous electrically conducting wire turned to form a plurality of loops. The two lateral wound winged-coil structures/lobes 10a and 10b are electrically connected in series by an intermediate wire segment 10c.

FIG. 1B shows the coil assembly 10 in a planar state wherein the winged-coil structures/lobes 10a, 10b are aligned in the same geometrical plane of their wound loops along an elongated axis (also referred to herein a deployment axis) 12y of the coil assembly 10. In some embodiments each loop of the winged-coil structures/lobes 10a, 10b of the coil assembly includes two layers of the loop windings. Particularly, each of the wound lobes 10a and 10b includes a plurality of circular loops, in this specific embodiment four loops are shown, spiraling outwardly from one or more innermost loops towards one more outermost loop. The loops are spiraling in such a way that each loop has a top winding and a bottom winding forming the two layers of the coil in each of the winged-coil structures/lobes 10a and 10b.

As seen in FIG. 1A, in some embodiments an intermediate wire segment 10c connects an innermost loop of the winged-coil structure/lobe 10a to an outermost loop of the other winged-coil structure/lobe 10b, to thereby provide that additive magnetic fields are generated by the two wings/lobes towards the TMS brain target of the treated subject. In some possible embodiments, the winged-coil structures/lobes of the coil assembly 10 are configured with some degree of flexibility, to thereby provide an adjustable wearable coil structure that can be snugly fitted to the shape of different curved portions/regions of the skull of the treated subject, as will be further described and exemplified hereinbelow with reference to FIGS. 1C to 1E.

As shown in FIG. 1B, adjacently located portions/elements of the two spaced apart winged-coil structures/lobes 10a and 10b define a central interaction zone ii wherein the additive electric and magnetic fields generated by the winged-coil structures/lobes components are of maximal strength. In this configuration the electric field generated by the winged-coil structures/lobes 10a, 10b of the coil assembly 10 are substantially tangential to the head of the treated subject (e.g., a selected portion of the skull) along a field application axis 10x, which in some embodiments is substantially perpendicular to the elongated/deployment axis 12y of the coil assembly 10, and substantially parallel to, or coincides with the tilt axis 12x of the winged-coil structures/lobes 10a and 10b of the coil 10. Such an arrangement of the winged-coil structures/lobes 10a and 10b provides that the tangential electric field is maximal along the field application axis 10x while all non-tangential components of the electric field are substantially attenuated and/or negligibly eliminated. This way the intensity of the induced field in the target brain region beneath the central segment i.e., the interaction zone ii, of the coil assembly 10 is increased and stimulation of desired neural structures of the brain can be ameliorated.

In operation, the angle between the winged-coil structures/lobes 10a and 10b is adjusted in accordance with the dimensions of the skull of the treated subject such that the winged-coil structures/lobes 10a and 10b are placed over upper portions of the head of the treated subject for positioning each wound winged-coil structure/lobe over upper lateral sides of the parietal bone. Electric currents are generated by a signal source (not shown) connected to the coil assembly 10 via the coil current terminals 11a and 11b. The electric currents passed through the winged-coil structures/lobes 10a and 10b generate electromagnetic fields directed to specific target brain regions of the treated subject to affect responsive electric stimulations therein.

Reference is made to FIGS. 1C and 1D, exemplifying treatment sessions applicable using the coil assembly 10 according to some possible embodiments. For the sake of simplicity, in these examples the coil assembly 10 is illustrated without the fastening arrangement (12 in FIGS. 1A and 1B), placed at different areas and orientations on a skull 17 of a patient. As seen, the configuration of the coil assembly 10 has sufficient flexibility to provide an adjustable wearable coil structure that can be fitted to the shape of different curved portions/regions of the skull of the treated subject such that substantial or all of the bottom side windings of the wings/lobes are in direct contact with the head 17 of the treated subject, to thereby maximize the strength of the electromagnetic field induced by the coil at the TMS target.

In particular, in FIG. 1C the winged-coil structures/lobes 10a, 10b of the coil 10 are placed at the central parietal portion of the skull 17, such that the elongated axis (12y in FIG. 1B) is substantially parallel to, or aligned in a coronal plane (i.e., horizontally) of the skull 17. As shown, the electric field is generated by the coil assembly 10 along the field application axis 10x, which is substantially parallel to, or aligned in the sagittal plane of the skull 17. The winged-coil structures/lobes of the coil assembly 10 can be secured to the head 17 of the treated subject by one or more straps 10k, as exemplified in FIG. 1D.

In FIG. 1D the elongated axis (12y in FIG. 1B) of the winged-coil structures/lobes 10a, 10b of the coil assembly 10 is substantially parallel to, or aligned in the coronal plane (i.e., horizontally) of the skull 17, such that one of the winged-coil structures/lobes 10a is located at the central parietal portion of the skull 17, while the other winged-coil structure/lobe 10b is located on a side of the parietal portion of the skull 17 e.g., for placing the interaction zone ii of coil assembly 10 approximately above the right hand motor cortex representation at the left hemisphere. In this arrangement the electric field is generated by the coil assembly 10 along the field application axis 10x in a plane slightly tilted with respect to the sagittal plane of the skull 17.

FIG. 1E shows a circular butterfly-shaped coil 10 locked by a coil fastening mechanism 29 in a planar state. The coil fastening mechanism 29 enables the locking of the wings 10a, 10b with a desired angle therebetween for snugly fitting to the head of the treated subject. In this specific and non-limiting example the coil fastening mechanism 29 comprises a respective lever 13a, 13b connected to a respective coil fastener structure 12a, 12b. As seen, the levers 13a, 13b can be coupled to a coil holder apparatus 41 via a respective strap 19a, 19b. The strap 19a, 19b is connected in some embodiments to the coil holder apparatus 41 via a respective releasable connector (e.g., buckle) 23, 24 rotatably coupled to the coil holder apparatus 41.

Each of the levers 13a, 13b comprises in some embodiments an attachment portion 13t configured for direct connection to the coil fastener structure 12a, 12b, and a slanted levering portion 13r extending upwardly from the attachment portion 13t for facilitating manual adjustment of the tilt angle between the winged-coil structures 10a, 10b. Optionally, but in some embodiments preferably the straps 19a, 19b are elastic straps configured to maintain a certain level of tension. Alternatively, or additionally, the straps 19a, 19b are adjustable straps configured for adjusting the loop size of the strap.

Each releasable connector (e.g., buckle) 23, 24 can have a male side 23m, 24m snappily connectable to a respective female side 23f, 24f. In some embodiments, as demonstrated in FIG. 1E, the male side 23m, 24m is connected to the respective strap 19a, 19b, and the respective female side 23f, 24f is rotatably connected to the coil holder apparatus 41. In other embodiments the male side 23m, 24m of the respective buckle 23, 24 is connected to the coil holder apparatus 41 and the female side 23f, 24f is coupled to the fastening means 12a, 12b attached to the coil assembly, such as via the straps 19a, 19b and/or the levers 13a, 13b. When the releasable connector 23, 24 is closed (i.e., the male 23m, 24m and female 23f, 24f sides are connected), the winged-coil structures 10a, 10b are locked in a flat planar state. When the releasable connector 23, 24 is opened (i.e., the male 23m, 24m and female 23f, 24f sides are separated), the winged-coil structures 10a, 10b are flexible and can be tilted and form various angles between them.

During a TMS procedure the coil may be placed over a subject head. On common procedure is location of the hot spot and determination of the motor threshold (MT), which is the minimal stimulator power output required to induce motor response of hand or foot muscle. The hot spot is the location and orientation of the coil over the motor cortex where the MT is minimal. Another procedure is movement of the coil from the hot spot on the motor cortex, to the treatment position, attachment of the coil at the treatment position and administration of TMS treatment session. In one embodiment all or part of the above procedures are done with the releasable connector 23, 24 closed, so that the winged-coil structures 10a, 10b are locked in a flat planar state. In another embodiment, all or part of the above procedures are done with the releasable connector 23, 24 opened, so that the winged-coil structures 10a, 10b are flexible and can be tilted and form various angles between them. In some embodiments the winged-coil structures 10a, 10b are made to conform a portion of the subject head.

Accordingly, the releasable connectors 23, 24 are configured to define a locked states of the coil fastening mechanism 29, and a released state thereof. In the released state, the releasable connectors 23, 24 are opened to facilitate adjustment of a suitable tilt angle between winged-coil structures 10a, 10b for snug fitting to the head of the treated subject. In the locked state, the releasable connectors 23, 24 are closed to maintain the tilt angle between the winged-coil structures 10a, 10b during the TMS treatment session, so as to prevent a further tilt between winged-coil structures 10a, 10b.

FIG. 1F shows a butterfly-shaped coil enclosed inside a case element 10s, with the buckle 23 which enables to lock the winged-coil structures at a planar state, and the head straps 32 which, once the buckle 23 is opened, enable to snugly fit and attach the winged-coil structures to the head of the treated subject in a state where the winged-coil structures conform to the head and assume a certain angle between them.

FIG. 1G shows a circular butterfly-shaped coil 10 attached to a human head 50. The buckles (23, 24 in FIG. 1E) are shown here in the open state, such that the winged-coil structures can assume various angles. As seen, the head strap 32 attaches the winged-coil structures to the head 50.

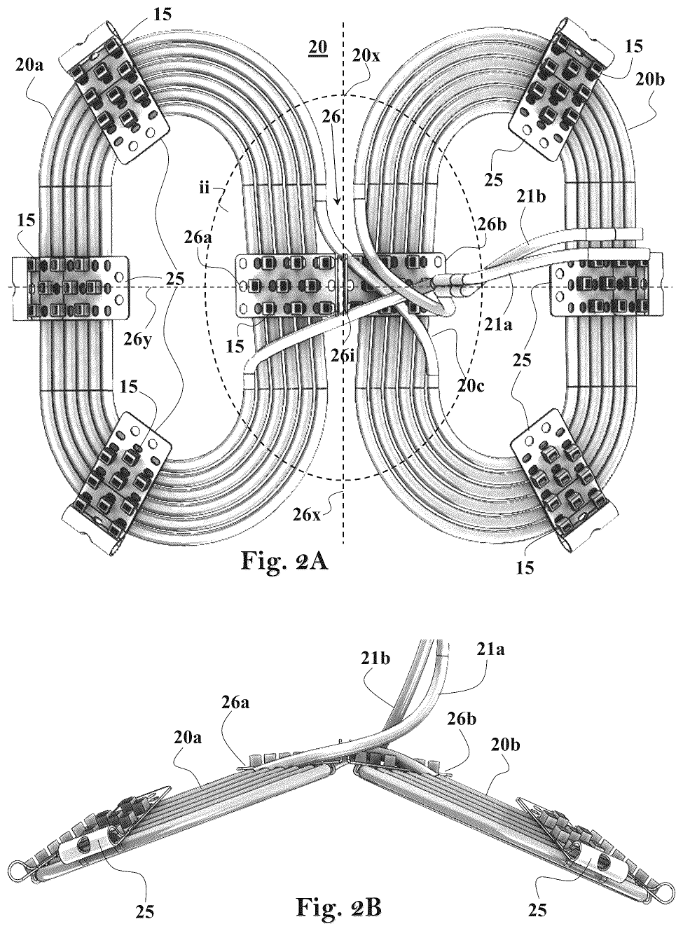

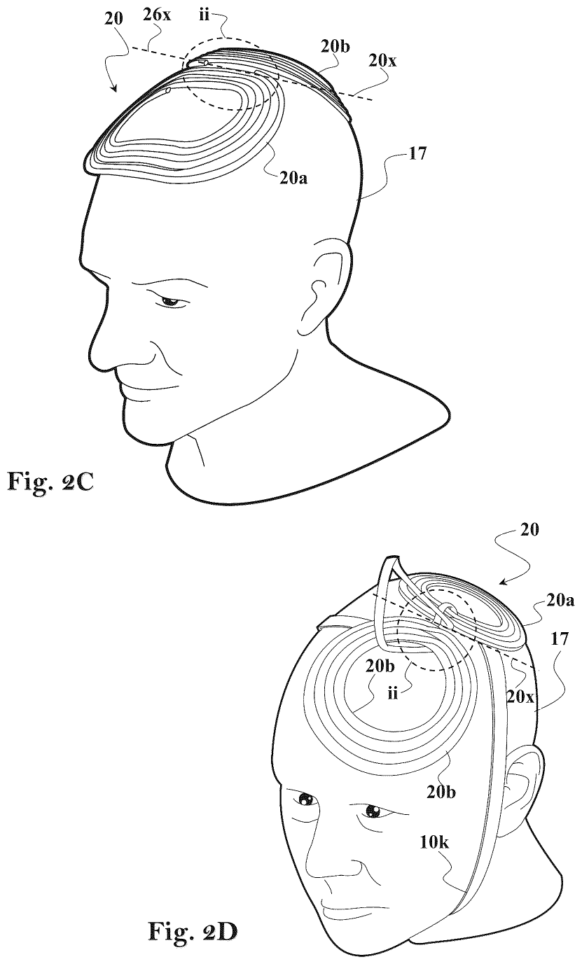

Reference is now made to FIGS. 2A and 2B, illustrating, respectively a top- and a side-view of a butterfly-shaped electromagnetic coil assembly 20 according to some possible embodiments. The electromagnetic butterfly-shaped coil assembly 20 includes two spaced apart ellipsoid-shaped winged-coil structures/lobes of loops, 20a and 20b arranged along extent/deployment axis 26y, wherein each winged-coil structure/lobe is forming a planar coil structure, and an adjustable/flexible fastening arrangement 26 connecting between the two ellipsoid-shaped winged-coil structures/lobes 20a and 20b. The fastening arrangement 26 is configured for easily varying an angle between planes of the two ellipsoid-shaped winged-coil structures/lobes 20a and 20b, so as to fit/conform the coil assembly 20 to the size/shape of the head/skull of the treated subject, while enabling elastic deformation of other portions of the winged-coil structures/lobes of the coil 20.

In some possible embodiments, the fastening arrangement 26 is formed by a pair of fastening plates 26a and 26b to which main portions/elements of the loops of the winged-coil structures/lobes 20a and 20b are respectively attached e.g., by a plurality/array of fasteners 15 (e.g., cable ties/zip ties). Optionally, and in some embodiments preferably, the fastening plates 26a and 26b are pivotally connected between them via hinge 26i configured to enable angular movement of the fastening plates 26a and 26b one with respect to the other about their tilt axis 26x. Optionally, but in some embodiments preferably, the tilt axis 26x is substantially perpendicular to the deployment axis 26y.

Accordingly, an angle between the fastening plates 26a and 26b can be manipulated, i.e., increased or decreased, so as to vary angular orientation of the ellipsoid-shaped wings/lobes 20a and 20b (planes thereof) with respect to each other. This way the coil assembly 20 can be readily adjusted to snugly fit/conform to the size of the curved head/skull of the treated subject such that substantial or all of the bottom side windings of the wings/lobes 20a, 20b are in direct contact with the head (17) of the treated subject.

The return portions/elements of the loops of the coil assembly 20 are attached e.g., by a plurality/array of fasteners 15 (e.g., cable ties/zip ties) to the fastening plates 25 distributed along segments of the loops of the coil 20 that are outside the interaction zone ii. The flexibility of the winged-coil structures/lobes 20a, 20b of the coil assembly 20 is this way improved by connecting the main portions/elements of the coil loops inside the interaction zone ii to a single fastening plates 26a, 26b, and connecting the return portions/elements of the coil loops that are relatively remote (with loop paths) by several fastening plates 25 (three in this non-limiting example). This way more than a quarter of unfastened loop length is obtained on each side of the fastening plates 26a, 26b inside the interaction zone ii, which enable flexible deformation of the return portions/elements of the loops outside the interaction zone ii with respect to the main portions/elements of the loops.

FIG. 2A shows the coil assembly 20 in a planar state, wherein its winged-coil structures/lobes are substantially aligned along the deployment axis 26y. FIG. 2B shows the coil assembly 20 in a slanted conformation in which an obtuse angle is formed between the winged-coil structures/lobes 20a, 20b of the coil as they are angularly tilted about the tilt axis 26x defined by the hinge 26i. Optionally, and in some embodiments preferably, the winged-coil structures/lobes of the coil assembly 20 are made from a single continuous electrically conducting wire turned to form the two lateral wound wings/lobes 20a and 20b electrically connected in series by an intermediate wire segment 20c electrically connecting the innermost loop of winged-coil structure/lobe 20b to the outermost loop of the wing/lobe 20a. The coil assembly 20 can be electrically coupled to a signals power source (not shown) via the coil's current terminals 21a and 21b, to generate an additive magnetic field by the winged-coil structures/lobes.

The coil assembly 20 also includes a plurality of auxiliary fastening plates 25 (three in each of the wings/lobes 20a and 20b in this specific configuration). The wires of the wings/lobes are connected to each one of the fastening plates 25 e.g., by a plurality/array of fasteners 15 (e.g., cable ties/zip ties) configured for holding together the loops of the winged-coil structures/lobes, as well as to maintain the planar geometry of the winged-coil structures/lobes 20a and 20b.

As shown in FIG. 2A, adjacently located portions/elements of the two spaced apart winged-coil structures/lobes 20a and 20b define an interaction zone ii in which the additive magnetic fields generated by the winged-coil structures/lobes are maximal. The electric field generated by the coil assembly 20 is tangential to the head of the treated subject (e.g., a selected portion of the skull) along a field application axis 20x which is substantially parallel to, or coincides with, the tilt axis 26x of the winged-coil structures/lobes 20a and 20b of the coil assembly 20. Such an arrangement of the winged-coil structures/lobes 20a and 20b provides that in the interaction zone ii the tangential component of the induced electric field is maximal while all non-tangential components of the induced electric field are substantially attenuated or negligibly eliminated. This way stimulation of desired target neural structures of the brain of the treated subject can be ameliorated.

FIG. 2C shows the electromagnetic coil assembly 20 in operation, placed on the head/skull 17 of a treated subject. As seen, the winged-coil structures/lobes 20a and 20b are placed at the central parietal portion of the skull 17 and arranged such that the tilt axis 26x of the coil assembly 20 is substantially parallel to, or aligned in a coronal plane (i.e., vertically) of the skull 17. FIG. 2D shows application of the coil assembly 20 such that the ellipsoid shaped winged-coil structures/lobes 20a, 20b are lateral to the sagittal plane of the head 17 of the treated subject, and their deployment axis (26y in FIG. 2A) is substantially parallel thereto e.g., to place the interaction zone ii of the coil 20 approximately above the right hand motor cortex representation at the left hemisphere.

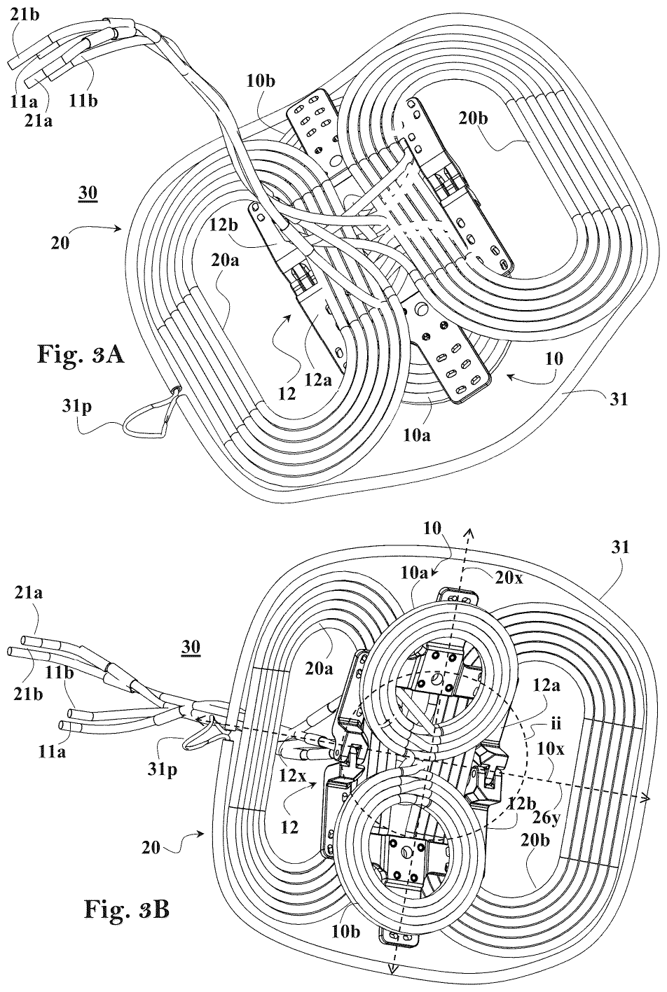

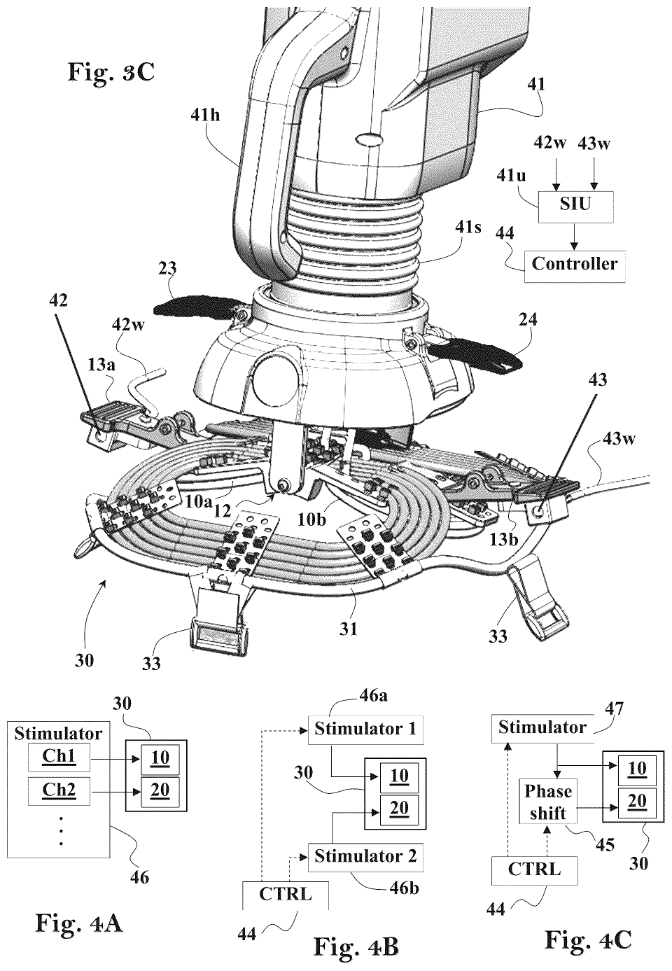

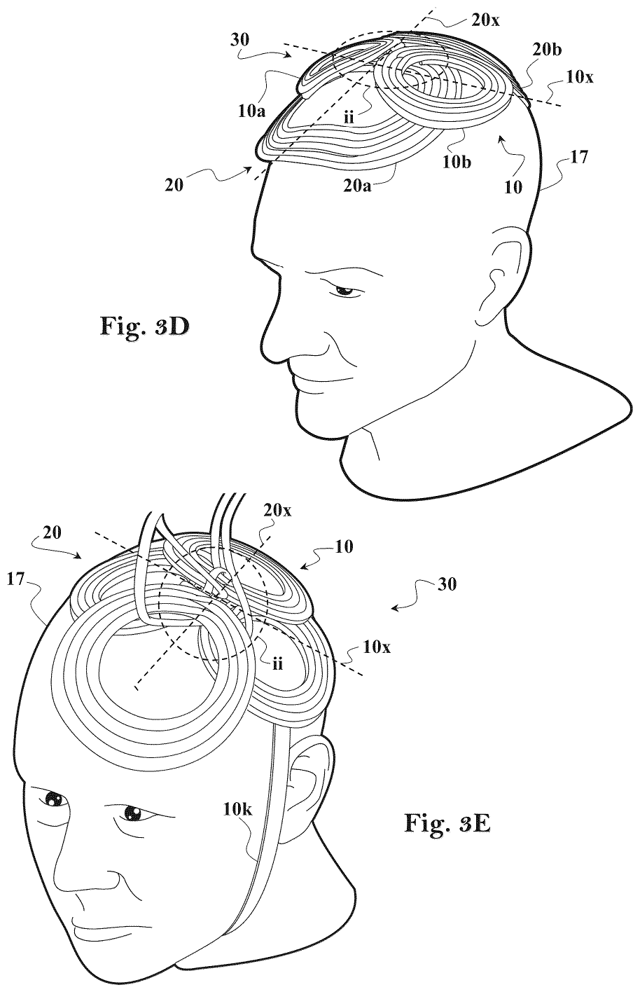

Reference is made to FIGS. 3A and 3B, illustrating, respectively a top- and a bottom-view of a combined electromagnetic coil array (also referred to herein as crossing coils structure) 30 according to some possible embodiments. The combined electromagnetic coil array 30 includes the butterfly-shaped coil assembly 10 shown in FIGS. 1A and 1B with the loops of its circular-shaped winged-coil structures/lobes 10a and 10b coupled to a bottom surface of the fastening arrangement 12, and loops of the ellipsoid-shaped winged-coil structures/lobes 20a and 20b of the coil assembly 20 shown in FIGS. 2A and 2B coupled to the opposite top surface of the fastening arrangement 12.

The coils 10 and 20 are arranged in the combined coil array 30 so as to form a defined angle (e.g., 90°) between the tilt axis (12x in FIG. 1B) of coil 10 and the tilt axis (26x in FIG. 2A) of the coil 20. Such crossing coils structure 35 can be similarly obtained by forming the defined angle between the deployment axis (12y in FIG. 1B) of coil 10 and the deployment axis (26y in FIG. 2A) of the coil assembly 20.

This crossing coils structures 30 enable setting a desired tilt angle between the winged-coil structures 10a, 10b and/or 20a, 20b of the coil assemblies 10 and 20, while guaranteeing that the central interaction zones ii of the coil assemblies 10 and 20 substantially coincide. Particularly, a desired tilt angle between the winged-coil structures 10a, 10b of the coil assembly 10 can be obtained by deforming the crossing coils structure 30 along the tilt axis 12x of the coil assembly 10, which may also at least partially deform the winged-coil structures 20a, 20b of the coil assembly 20 along its deployment axis 26y.

In some possible embodiments, the ellipsoid-shaped winged-coil structures/lobes 20a and 20b are accommodated on the top surface of the fastening arrangement 12 in a plane substantially parallel to the plane of the circular-shaped winged-coil structures/lobes of loops 10a and 10b. It is noted that terms such as top and bottom are used herein to indicate orientation of the coil assemblies disclosed herein in operative states. In this embodiment the bottom surface of the fastening arrangement 12 faces the skull/head of the treated subject and the circular-shaped winged-coil structures/lobes 10a and 10b placed over/on the skull/head of the treated subject while the ellipsoid-shaped winged-coil structures/lobes 20a and 20b are located slightly above the skull/head of the treated subject.

As better seen in FIG. 3B, this configuration of the combined coil 30 maintains the flexibility of the return elements/portions of the loops of the coil assemblies 10 and 20, allowing flexible deformation thereof with respect to the main portions/elements of the loops that are located withing the interaction zone ii of the combined/crossing coils structure 30. This way the geometrical shape of the combined/crossing coils structure 30 can be adjusted to conform to the portion of the head of the treated subject on which it is positioned such that substantial portion of the loops are in direct contact with the head of the subject, while guaranteeing that the central interaction zones ii of the coil assemblies 10 and 20 are substantially coinciding.

In some embodiments, the ellipsoid-shaped winged-coil structures/lobes 20a and 20b of coil assembly 20, and the circular-shaped winged-coil structures/lobes 10a and 10b of coil assembly 10 are electrically powered by a multi-channel TMS stimulator 46, as shown in FIG. 4A. Optionally, and in some embodiments preferably, the combined electromagnetic coil array 30 is controllably operated to induce a desired stimulation pattern. More specifically, the circular-shaped winged-coil structures/lobes of loops 10a, 10b of the coil assembly 10 can be powered by a first alternating current feed e.g., sequence of electric current pulses, and the ellipsoid-shaped winged-coil structures/lobes of loops, 20a, 20b of the coil assembly 20 can be powered by a second alternating current feed e.g., sequence of electric current pulses, configured such that the first and second electric current feeds have a predetermined phase shift between them e.g., a phase shift of π/2. This way, a rotating electromagnetic field can be generated which is maximal in the coinciding interaction zones ii of the coil assemblies 10 and 20.

Alternatively, two different stimulators 46a and 46b can be used to separately generate the first and second electric current feed for the coil assemblies 10 and 20, as exemplified in FIG. 4B. Optionally, a controller 44 is used to regulate the first and second electric currents feed driving the coil assemblies 10 and 20, and guarantee that a desired phase difference is maintained therebetween. Alternatively, a single-channel stimulator 47 is used to generate an electric feed current for one of the coil assemblies 10 (or 20), and a phase shift unit 45 is used to receive the electric feed current from the stimulator 47 and generate therefrom another electric feed current for the other coil 20 (or 10) having the desired phase difference, as exemplified in FIG. 4C. A controller 44 is optionally used to control the phase shift unit 45, and/or the stimulator 47, to guarantee that the desired phase difference between the two electric feed currents is maintained.

In some embodiments, only the circular-shaped winged-coil assemblies/lobes 10a and 10b of coil assembly 10, or the ellipsoid-shaped winged-coil structures/lobes 20a and 20b of coil assembly 10 are activated by an electric supply current, if so needed.

When the coil assemblies 10 and 20 of the combined/crossing coils array 30 are powered by two different alternating electric currents with a phase shift of about 90°, the magnetic field thereby generated in the coinciding interaction zones ii is of maximal strength, and the electric fields thereby generated are rotating tangential to the head of the treated subject (e.g., a selected portion of the skull). This configuration of the combined/crossing coils assembly 30 provides that in the coinciding interaction zones ii of the coil assemblies 10 and 20 the magnetic field generated by the combined/crossing coils array 30 is maximal, and that all non-tangential components of the field thereby generated are substantially attenuated. It is thus expected that a considerably lower motor threshold is obtained using the combined/crossing coil array 30, compared to the conventional flat figure-8 coils.

In possible embodiments combined/crossing coil structures such as illustrated in FIGS. 3A to 3C are prepared with a plurality (more than two) butterfly-shaped coils, such that a desired angle is obtained between the deployment angle of each butterfly-shaped coil and the deployment angle of at least another one of the plurality of butterfly-shaped coils. For example, in possible embodiments the combined/crossing coils structure can be configured to form an equiangular TMS coil structure, wherein the angle between the deployment axes of each pair of adjacently-located butterfly-shaped coils is substantially 180°/n, wherein n≥2 is the (integer) number of crossing coils in the TMS coil structure.

In some embodiments, the combined/crossing coil array 30 is coupled to a fastening frame structure 31 encircling the ellipsoid-shaped wings/lobes 20a and 20b. A gripping loop 31p may be provided in the fastening frame structure 31 to facilitate placement and adjustment of the coil array 30 on the head of the treated subject (17). One or more strap fasteners 33 may attached to the fastening frame structure 31 for securing the coil array 30 to the head of the treated subject e.g., by the head straps.

FIG. 3C shows the coil array 30 engaged in a holder apparatus 41 configured to facilitate placement of the coil array 30 on the head of the treated subject. In this non-limiting example the coil array 30 is equipped with sensor elements 42 and 43 (e.g., EM sensors) coupled at opposing lateral sides of the fastening arrangement 12 for electromagnetic neuronavigation. The sensor elements 42 and 43 can be coupled to the combined/crossing coils array 30 by attaching them to the levers 13a and/or 13b.

The sensor elements 42 and 43 are used together with other sensor elements (not shown) placed on the head of the treated subject to accurately position the combined/crossing coil array 30 over a target TMS region of the head of the treated subject. The holder apparatus 41 can be equipped with an electromagnetic (EM) field generator or transmitter (not shown) configured to emit a low-intensity varying EM field that establishes the measurement volume sensed by the sensor elements. In operation, small currents are induced in the sensor elements 42, 43 when they enter the generated EM field. The induced currents are relayed via wires 42w and 43w to the sensor interface unit (SIU) 41u configured to amplify and digitize the signals thereby received, and transmit the same to the system control unit (SCU) 44.

The control unit 44 is configured to calculate for each sensor element 42, 43 a position and orientation (e.g., as a transformation) based on the received signals and the geometrical position of the sensors 42 and 43 with respect to the coil assemblies 10 and 20 and the other sensor elements placed on the head of the treated subject. The control unit 44 then generates instructions for the user to move the combined/crossing coil array 30 a determined displacement in a suitable direction for locating the coil assemblies over the desired target brain region of the treated subject. The holder apparatus 41 is further configured to connect the coil assemblies 10, 20 of the combined/crossing coil array 30 to one or more stimulators (e.g., as exemplified in FIGS. 4A to 4C), and stream a cooling media through coolant conduit 41s towards the coil assemblies 10, 20 to prevent overheating. A handle 41h is provided in some embodiments to facilitate placement of the holder apparatus 41 and the combined/crossing coils array 30 on the head of the treated subject.

FIG. 3D shows the combined/crossing coil array 30 placed on the head 17 of the treated subject readily operative for application of electromagnetic fields thereinto. In the combined/crossing coils configuration shown in FIG. 3D the elliptical butterfly-shaped winged-coil structures/lobes 20a, 20b of the coil assembly 20 are placed in direct contact with the head of the treated subject, and the circular winged-coil structures/lobes 10a, 10b of the coil assembly 20 are located on top of the winged-coils structures/lobes of coil assembly 20. The combined/crossing coils array 30 is shown in FIG. 3D without the fastening arrangement 12, and without the holder apparatus 41 for the sake of simplicity.

Due to the lateral flexibility of the elliptical butterfly-shapes winged-coil structures/lobes 20a, 20b they are arranged at a medial position with substantial portion, or all, of the bottom sides loops of the winged-coil structures/lobes 20a, 20b conforming to the shape of the head and in direct contact therewith.

FIG. 3E shows the combined/crossing coils array 30 attached to the head 17 of the treated subject at a lateral position, wherein the interaction zone of the combined/crossing coils array 30 is approximately above the right hand motor cortex representation at the left hemisphere. As seen, the tilt angle between the circular winged-coil structures/lobes 10a, 10b of the bottom side coil assembly 10 is adjusted to bring substantial portion, or all, of the bottom side loops to directly contact the head 17 of the treated subject. Also seen in FIG. 3E, due to the flexibility of the winged-coil structures/lobes 20a, 20b of the upper-side of the coil assembly 20, their lateral portions substantially conform to the shape of the head 17 and thereby in direct contact therewith.

The winged-coil structures/lobes of the coil assemblies 10, 20 of the combined/crossing coils array 30 can be secured to the head 17 of the treated subject by one or more head straps 10k.

It should also be understood that throughout this disclosure, where a process or method is shown or described, the steps/acts of the method may be performed in any order and/or simultaneously, and/or with other steps/acts not-illustrated/described herein, unless it is clear from the context that one step depends on another being performed first. In possible embodiments not all of the illustrated/described steps/acts are required to carry out the method.

Relative terms such as “lower,” “upper,” “horizontal,” “vertical,” “above,” “below,” “up,” “down,” “top” and “bottom”, as well as derivatives thereof (e.g., “horizontally,” “downwardly,” “upwardly,” etc.), and similar adjectives in relation to orientation of the described elements/components refer to the manner in which the illustrations are positioned on the paper, not as any limitation to the orientations in which these elements/components can be used in actual applications.

As described hereinabove and shown in the figures, the present application provides flexible/deformable TMS coils for efficient application of electromagnetic fields in brain regions of a treated subject, and related methods. While particular embodiments of the invention have been described, it will be understood, however, that the invention is not limited thereto, since modifications may be made by those skilled in the art, particularly in light of the foregoing teachings. As will be appreciated by the skilled person, the invention can be carried out in a great variety of ways, employing more than one technique from those described above, all without exceeding the scope of the claims.

1. A TMS coil assembly for magnetic stimulation of a brain region of a treated subject, the TMS coil assembly comprising:
one or more coil assemblies, each one of said coil assemblies comprising adjacently located, or spaced-apart, flexible winged-coil structures configured to adjustably deform along a tilt axis passing between said winged-coil structures to apply a desired tilt angle between the winged-coil structures for them conform to curved surfaces of a head of a treated subject and bring substantial loop portions of said winged-coil structures into direct contact with said curved surfaces of the head, to thereby maximize the loop portions that are tangential to said curved surfaces of the head and minimize the loops portion that are non-tangential to said curved surfaces; and
a fastening arrangement configured to maintain the desired tilt angle between the winged-coil structures when placed on the head of the treated subject during a TMS procedure, to thereby maximize strength of electric field thereby induced into said head.

2. The TMS coil assembly of claim 1 configured to define a field application axis passing between the winged-coil structures.

3. The TMS coil assembly of claim 2 wherein the tilt axis and the field application axis are substantially parallel or coincide.

4. The TMS coil assembly of claim 2 or 3 configured to define a central interaction zone, with respect to the field application axis, in which additive electric and magnetic fields generated by the winged-coil structures are of maximal strength.

5. The TMS coil assembly of claim 1, wherein the desired tilt angle is a flat angle.

6. The TMS coil assembly of claim 1 wherein the fastening arrangement comprises one or more coil retaining structures attached to the winged-coil structures and configured to facilitate adjustment of the tilt angle.

7. The TMS coil assembly of claim 6 wherein the one or more coil retaining structures are hinged with one or more pivots.

8. The TMS coil assembly of claim 6 comprising one or more levering members each coupled to a coil holder apparatus and to at least one of the coil retaining structures to facilitate setting of the desired tilt angle between the winged-coil structures.

9. The TMS coil assembly of claim 8 comprising a locking mechanism configure to maintain the levering members locked with respect to the coil holder apparatus so as to prevent further tilting of the winged-coil structures.

10. The TMS coil assembly of claim 9 comprising one or more releasable connectors, each configured to couple one of the levering members to the coil holder apparatus.

11. The TMS coil assembly of claim 10 wherein each releasable connector is connected to a respective levering member by a respective elastic and/or adjustable strap.

12. The TMS coil assembly of claim 1 comprising securing means configured to attach said TMS coil assembly to the head of the treated subject.

13. The TMS coil assembly of claim 12, wherein the securing means comprises head straps configured to secure said TMS coil assembly to the head and bring substantial portion of their loops into direct contact therewith.

14. The TMS coil assembly of claim 1 configured with several degrees of flexibility of the winged-coil structures for allowing the loops of the coil to conform to the head of the treated subject and bring substantial portions of the loops into direct contact therewith.

15. The TMS coil assembly of claim 1 wherein the loops of the winged-coil structures are configured to flexibly conform and contact the head in all of the coil portion facing the head.

16. The TMS coil assembly of claim 1 wherein the winged-coil structures have a generally circular shape.

17. The TMS coil assembly of claim 1, wherein the winged-coil structures have a generally elliptical shape.

18. The TMS coil assembly of claim 1, wherein the winged-coil structures have a generally rectangular shape.

19. A The TMS coil array for magnetic stimulation of a brain region of a treated subject, the coil array comprising two or more of the TMS coil assemblies of claim 1, each one of said TMS coil assemblies having a deployment axis being substantially perpendicular to its tilt axis, and wherein the deployment axis of each one of said two or more TMS coil assemblies crosses the deployment axis of at least another one of said TMS coil assemblies.

20. The TMS coil array of claim 19 comprising two of the TMS coil assemblies connected to two different sides of a single fastening arrangement.

21. The TMS coil array of claim 20, wherein an angle between the tilt axes of the TMS coil assemblies is about 90 degrees.

22. The TMS coil array of claim 19 wherein each one of the two or more TMS coil assemblies has a central interaction zone as defined in claim 4, and wherein said TMS coil array is configured such that the interaction zones of the two or more TMS coil assemblies substantially coincide.

23. The TMS coil array of claim 19, wherein each one of the TMS coil assemblies is electrically connected to a respective independent channel of a stimulator device configured for controlling the timing of operation of said TMS coil assemblies.

24. The TMS coil array of claim 19 configured for operation of a phase difference between electric currents of at least two of the TMS coil assemblies, to thereby induce a rotating field in the brain tissue.

25. The TMS coil array of claim 19 wherein each or some wires of the TMS coil assemblies are electrically insulated separately.

26. The TMS coil array of claim 19 comprising one or more layers of an electric insulation shield surrounding all wires of the TMS coil assemblies.

27. The TMS coil array of claim 19 configured for neuronavigation of position of the coil assemblies on the head.

28. The TMS coil array of claim 27 wherein the neuronavigation is based on optical and/or electromagnetic tracking.

29. A method for treating a neurophysiological condition, the method comprising:
placing at least one TMS coil assembly having winged-coil structures on a head portion of a treated subject;
setting a tilt angle between said winged-coil structures for them to conform to curved surfaces of the head so as to bring substantial portions of their loops into direct contact with the head; and
passing electric currents in said at least one TMS coil assembly to stimulate a region of the brain of the treated subject.

30. The method of claim 29 comprising changing the TMS coil assembly into a released state before the setting of the tilt angle, and thereafter changing the TMS coil assembly into a locked state for preventing further tilting between the winged-coil structures.

31. A The method for treating a neurophysiological condition, the method comprising:
placing the TMS coil assembly of claim 1, on a head of a treated subject and setting a tilt angle between at least two winged-coil structures thereof so as to conform to a curved surface area of said head; and
passing electric currents in the TMS coil assemblies of said TMS coil array to stimulate a region of a brain of said subject.

32. The method of claim 31, wherein said neurophysiological condition is at least one of the following: depression, bipolar disorder, schizophrenia, PTSD, Parkinson's disease, dystonia, movement disorder, Alzheimer's disease, mild cognitive impairment, autism, Asperger's syndrome, multiple sclerosis, ALS, Tourette's syndrome, blepharospasm, stroke, chronic pain, eating disorder, obesity, anorexia nervosa, bulimia, any addiction including smoking addiction, drug addiction, alcoholism or gambling, ADHD, OCD, epilepsy, migraine, tinnitus.

33. The method for treating a neurophysiological condition, the method comprising:
placing TMS coil array of claim 19, on a head of a treated subject and setting a tilt angle between at least two winged-coil structures thereof so as to conform to a curved surface area of said head; and
passing electric currents in the TMS coil assemblies of said TMS coil array to stimulate a region of a brain of said subject.

34. The method of claim 33, wherein said neurophysiological condition is at least one of the following: depression, bipolar disorder, schizophrenia, PTSD, Parkinson's disease, dystonia, movement disorder, Alzheimer's disease, mild cognitive impairment, autism, Asperger's syndrome, multiple sclerosis, ALS, Tourette's syndrome, blepharospasm, stroke, chronic pain, eating disorder, obesity, anorexia nervosa, bulimia, any addiction including smoking addiction, drug addiction, alcoholism or gambling, ADHD, OCD, epilepsy, migraine, tinnitus.
